(19) United States
(12) Patent Application Publication
(10) Pub. No.: US 2005/0170354 A1
(43) Pub. Date: Aug. 4, 2005

(54) Process for centrifugal distribution of liquid physiological specimens

(75) Inventor: Jakob Munch-Fals, Virum (DK)

Correspondence Address: Richard R Michaud;McCormick Paulding & Huber, City Place II, 185 Asylum Street, Harford, CT, 06103, US

(73) Assignee: Danfoss A/S, Nordborg (DK)

(21) Appl. No.: 10/510,559

(22) PCT Filed: Mar. 27, 2003
(86) PCT No.: PCT/DK03/00209

(30) Foreign Application Priority Data
Apr. 9, 2002 (DK) PA 2002 00514

(51) Int. Cl. C12Q 1/68; B04B 5/02; C12N 1/08

(52) U.S. Cl. 435/6; 435/270; 494/16

(57) ABSTRACT

This invention discloses an improved process of distributing liquid physiological specimens over a surface, i.e. the filming process in a laboratory, during which a liquid sample is distributed over a specimen-display surface like a conventional laboratory slide. The invention relates to the utilisation of the centrifugal force in a rotating device. By placing the surface that is to be filmed by the sample, in such a way that it is pointed towards the axis of rotation, the influence of the centrifugal force will distribute the sample over the whole surface.

### CROSS-REFERENCE TO RELATED APPLICATIONS

This application is entitled to the benefit of and incorporates by reference essential subject matter disclosed in International Application No. PCT/DK03/00209 filed on Mar. 27, 2003 and Danish Patent Application No. PA 2002 00514 filed on Apr. 9, 2002.

### FIELD OF THE INVENTION

This invention relates to a process for centrifugal distribution of liquid physiological specimens over a surface with an array of assays, the array being placed in a rotating device and the liquid physiological specimens being distributed on the surface by the dynamic forces of the rotation, the surface pointing towards the axis of rotation, the liquid physiological specimens being forced towards the surface under the influence of the dynamic forces of the rotation.

### BACKGROUND OF THE INVENTION

Centrifugal distribution of liquids physiological specimens over a surface with an array of assays is often referred to as “filming” by those working in medical laboratories, and the liquid physiological specimens are often called “samples”.

In this connection, the term “a liquid physiological specimen” is to be understood in a very broad manner, covering, of course, physiological specimens like blood and spittle, but also pre-treated DNA extracts are comprised by the term.

As an example, applying a drop of blood to a slide, where the surface of the slide contains an array of assays, takes place in a DNA-testing process. When the drop of blood is distributed over the surface of the slide, the elements in the blood will react or connect to the assays where they fit, and analysing the slide afterwards will give the result of the DNA-testing.

To achieve the contact between the elements of the sample and the assays, the elements have to diffuse from the sample to the assays, and obviously the duration of this diffusion will increase with the thickness of the sample layer on the surface of the slide. This will reduce the duration of the process, if the thickness of the layer is reduced, and therefore reducing the thickness of the layer is desirable.

When filming, the main problem to solve is to equally distribute and re-circulate the sample over the whole of the surface, the sample being distributed to all the assays in the array. One way of solving this problem is to place the array of assays flat in a rotating device, the surface being in the same plane as the rotational movement. This will force, or rather throw, the sample along the surface, due to the dynamic reaction to the rotation. In the following, this dynamic reaction will be referred to as the centrifugal force, and must be understood as the act of a particle moving away from the centre of a rotating movement.

When distributing a sample by throwing it across the surface of the array of assays, the sample applied to the surface does not distribute equally over the whole surface, reproduction of the result being hard to obtain. A single drop of the sample is not likely to distribute over the whole surface, and therefore does not necessarily reach all assays in the array. Therefore, a number of extra drops is applied to the surface, which will increase the duration of each test and the used amount of the sample, which is often expensive. Also, each specimen in the sample will be forced along the surface, contact between the specimens and all the assays in the array being hard to obtain, which also influences the reproduction ability of the test.

Another known method of overcoming the problems of the filming process is to force the sample through micro channels, in which the array of assays is contained. This method will reduce the duration of the process, but is very sensitive to pollution, the size of the elements in the sample, and the production of the micro channels.

It is an object of this invention to overcome the difficulties of reproducing test results. It is a further object of this invention to reduce the duration of each test, and to reduce the used amount of the sample for each test. Yet another object of the invention is to reduce the sensitivity with regard to pollution and to the size of the elements in the sample.

### SUMMARY OF THE INVENTION

The object of this invention is achieved in that the liquid physiological specimens are applied as drops to an area of the surface opposite a drain end, and in that the distribution of the liquid physiological specimens is controlled by adjusting the position of the surface relative to the axis of rotating, in such a way that a thin film of the liquid physiological specimens will be formed over the whole surface before drops are forced over the edge in the drain end of the surface. Hereby it is achieved that the elements in the sample are forced in the direction of the assays, and a drop of the sample will be completely filmed by the dynamic forces. Further, it is achieved that all assays in the array are in contact with the sample, and that the elements in the sample will have equal and best conditions for diffusion to the assays. Yet further, it is achieved that it is possible to control the direction of the distribution, and the rate at which the sample distributes over the surface.

It is an advantage that the array of assays itself further rotates around another axis than that of the rotating device, meaning that the elements in the sample will be distributed to all, or nearly all, assays in the array.

It is particularly preferred that the surface, to which the sample is applied, is formed as a hollow in a material part, where the hollow forms a closed container when the material part is covered with a lid, and where the sample is applied to the surface before the lid covers the hollow. The sample is contained in a closed container, and can be re-circulated over the surface without having any disturbing and/or damaging contact with the surroundings.

### BRIEF DESCRIPTION OF THE DRAWINGS

In the following, the invention will be described in detail with references to the drawings, showing:

FIGS. 1A-G illustrate a slide having a number of assays, and show the progression of a liquid sample under centrifugal force.

FIG. 2 is a partly in section perspective view of the rotating device of the present invention.

FIG. 3 is a partly in section perspective view of a portion of the rotating device of FIG. 2.

FIG. 4 shows an embodiment of an array of assays, placed in a hollow.

FIG. 5 is a perspective view of a device containing two arrays of assays, placed in a hollow.

FIG. 6 schematically illustrates a rotating array of assays in accordance with the invention.

FIGS. 7A-E are functional views showing the process or re-circulation in accordance with FIG. 6.

FIG. 8 schematically illustrates a embodiment of a rotating device.

FIG. 9 schematically illustrates a embodiment of a rotating device.

### DETAILED DESCRIPTION OF THE PREFERRED EMBODIMENTS

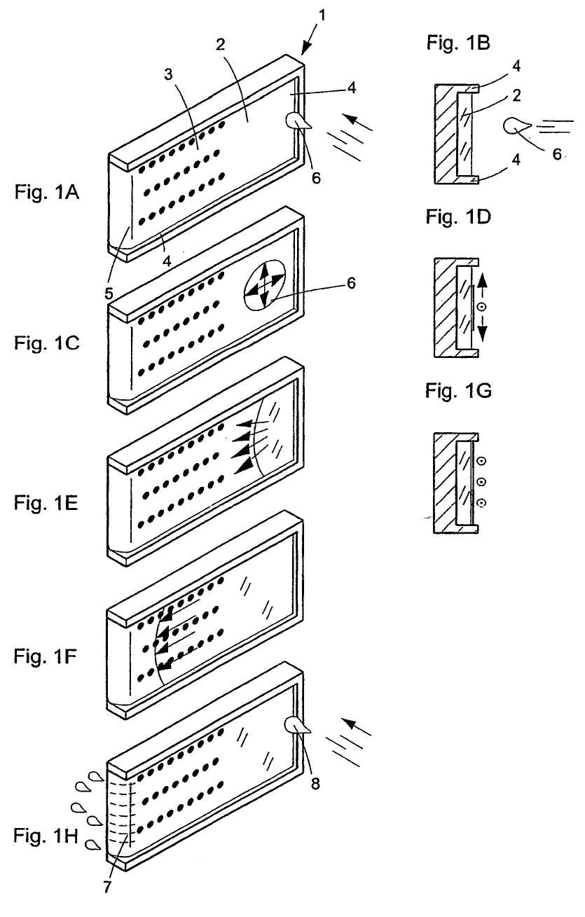

FIG. 1A shows a sectional view of a slide 1, having a surface 2 with an array of assays 3. Such slides are widely used in laboratories, e.g. for microscopic analyses of blood specimens. The slide of FIG. 1A has a frame part 4 along three of the sides, which will be understood from the sectional view in FIG. 1B. The fourth side 5 is formed and works as a drain part, where the specimens can flow away from the surface 2.

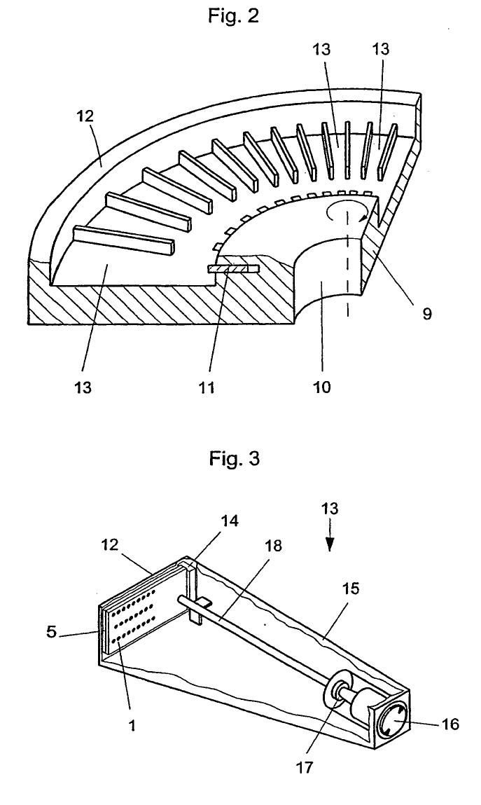

The slide 1 is placed in a rotating device 9 of FIG. 2, said device comprising several sections 13. FIG. 3 shows one of the sections 13 in a sectional view, and with the slide 1 placed in the holder 14. As the rotating device is rotating, centrifugal forces will act from the centre of rotation, and at right angle, towards the circumference 12 of the rotating device 9. The plunger part 11 of FIG. 2 will be forced towards the part 16 of FIG. 3, due to the dynamic forces, and under this pressure liquid sample contained behind part 16 will be dosed as drops by a capillary part 17, and led through the pipe 18 to the slide 1. The mouth of the pipe ends at the end of the slide where the holder is placed, and at a certain distance from the slide. The slide itself is placed in an upright position along the circumference 12 of the rotating device 9, the centrifugal force acting substantially parallel to a normal axis of the surface of the slide. The surface of the slide 1 is therefore substantially parallel to the rotating axis of the rotating device.

The drop 6 in FIGS. 1A and 1B is a drop, which has just left the pipe 18, and as the drop hits the surface of the slide, it will be distributed over the surface by the centrifugal force. This is indicated by arrows in FIGS. 1C to 1F, and the frame 4 on the three sides of the slide 1 will force the distribution towards the drain part 5 of the slide 1, from where drops 7 will be thrown of the slide. The centrifugal force has now distributed a thin layer of the sample over the whole surface, and every assay 3 in the array has got in contact with the sample.

The velocity with which the sample is distributed towards the drain part 5 can be controlled by controlling the angular position of the slide, and by controlling the rotational speed of the rotating device. As the distribution over the surface is controllable, only a very small amount of sample liquid is needed for each slide. As a consequence of the dynamic forces, surface tensions will only have limited effect.

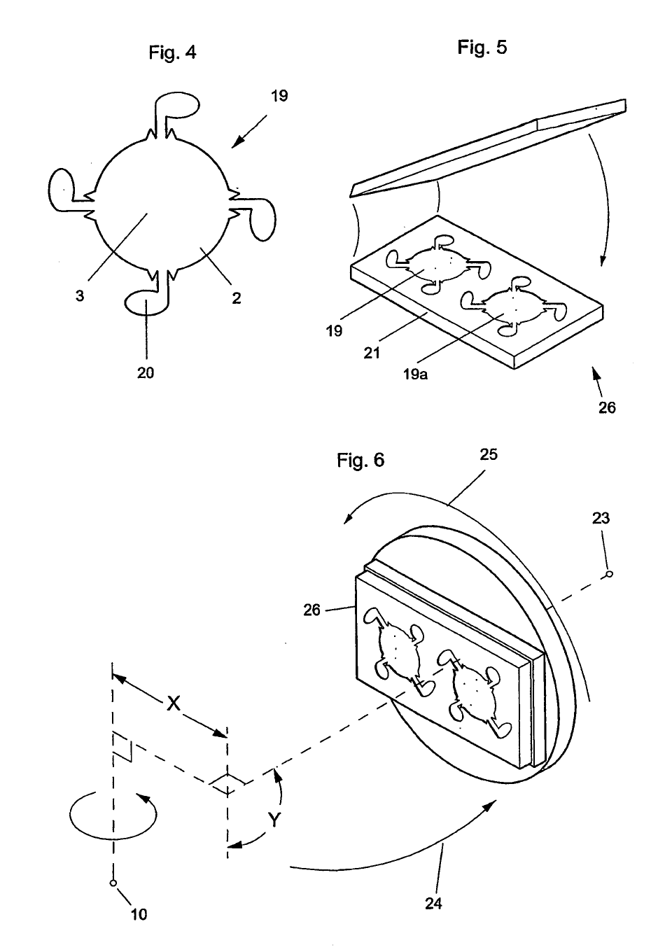

In some test application it is important that the used amount of sample liquid is reduced to a minimum, and that the applied sample liquid is maintained on the surface of the slide, and not thrown away over a drain part as previously described. This is known as re-circulation of the sample along the surface. FIG. 4 shows a structure 19, placed in a hollow, which has a surface 2 with an array of assays 3, and is supplied with small pockets 20 placed along the circumference of the structure. This structure is formed as a hollow 19 in a material part 21, shown in FIG. 5, with a lid 22 on top of it. When the lid is placed on top of the material part 21, each of the two hollows 19 and 19a forms a small test volume, and the assembled material part with lid forms a slide 26.

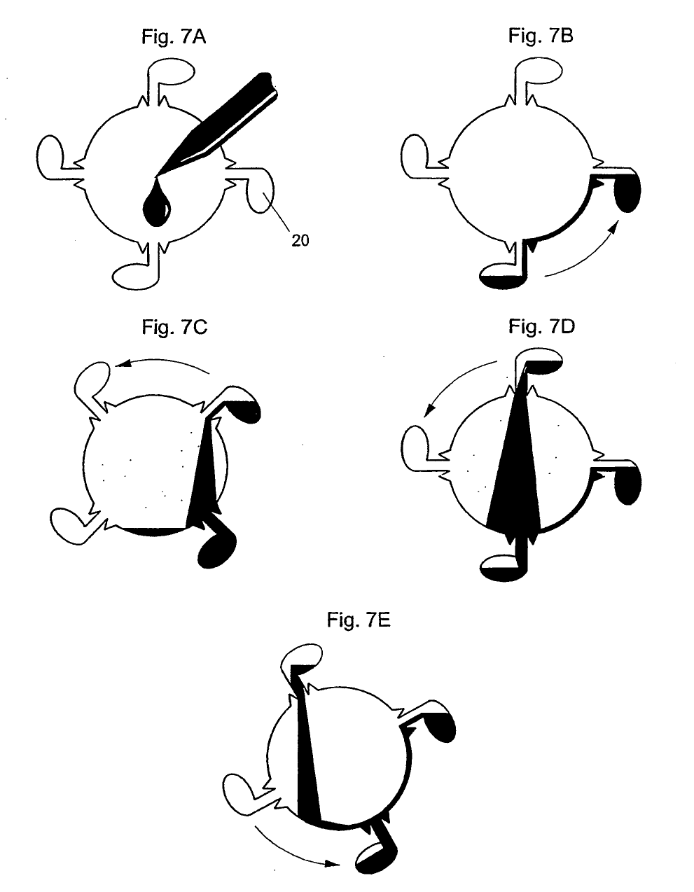

FIG. 6 shows the slide 26 placed in a rotating device with an axis of rotation 10, the rotation direction being indicated by an arrow 24. The slide 26 is placed at a centre line 23 that is placed at an angle Y relative to the rotating axis 10, and at a distance X from the rotating axis 10, the centrifugal force acting on the surface as previously described with a normal portion. In addition, the dynamic forces will act upon the surface with a tangential portion, the sample being forced across the surface. The slide 26 is rotated around the centre line 23, indicated by the arrow 25. The effect of this additional rotation 25 will be described with reference to FIG. 7.

FIG. 7A shows a drop of a liquid sample applied to the test volume in the container, and the lid is then placed on top of the container. Subsequently, the slide 26 is placed in the rotating device, the centrifugal force acting upon the surface and filming the sample on the array of assays. Some of the sample will be distributed into the small pockets, and as the slide 26 rotates around an axis, the liquid sample will flow from one pocket to the array of assays, and along the circumference to the next pockets, indicated in FIG. 7B. The shape of the pockets will, however, distribute liquid sample over the whole array of assays, as the slide 26 is rotated, indicated in FIGS. 7C to 7E. Each pocket then acts as a collecting area, from where liquid sample once again is distributed to the array of assays, and re-circulation occurs.

Re-circulation as described in FIG. 7 has basically the effect known from a washing machine. The sample is again and again washed across the array of assays, whereby reproduction of the testing is obtained.

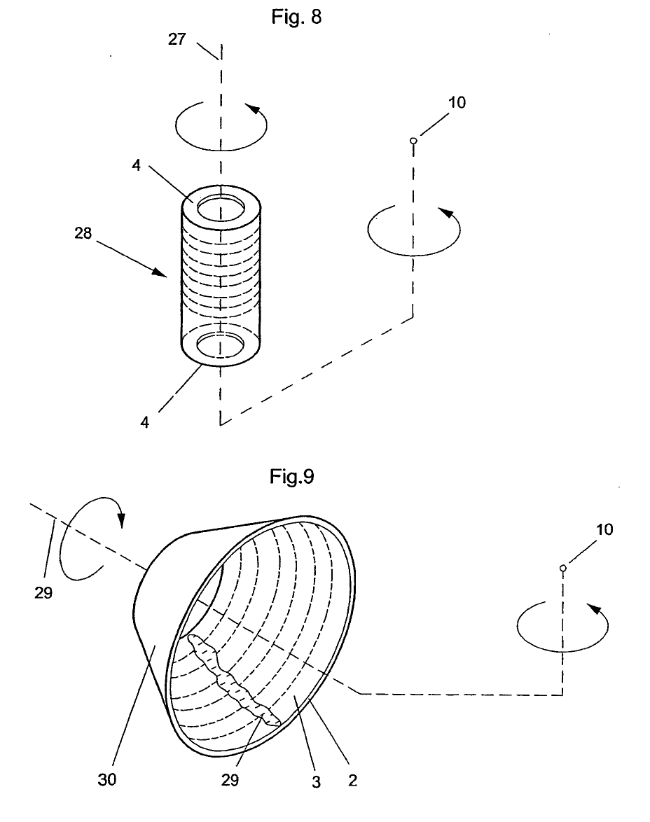

FIG. 8 shows another embodiment of a rotating slide in a rotating device. The slide 28 is now formed as a cylinder part, which rotates around its own axis 27. The axis 27 is more or less parallel to the rotation axis 10 of the rotating device, and the centrifugal force will thus act upon the surface of the slide 28. The array of assays is placed on the inner surface of the slide 28, and on the top and on the bottom the slide has a frame part 4, extending from the inner surface of the cylinder towards the axis of rotation 27. As a drop of the liquid sample is applied inside the slide 28, it will be distributed as a column along the inner surface, and the column will be placed where the distance from the axis of rotation 10 is largest. As the slide is rotated around the axis 27, the column will wash the whole of the inner surface, and hereby re-circulation will occur.

FIG. 9 shows a third embodiment of a rotating slide in a rotating device. Here the slide is formed as a cone part 30, which rotates around its own axis 29. The axis 29 is placed at an angle relative to the rotation axis 10 of the rotating device, a part of the inner surface of the cone shaped slide being more or less parallel to the rotation axis 10 of the rotating device, and the centrifugal force will thus act upon this part of the inner surface of the cone shaped slide 30. Due to the centrifugal force, a column of liquid sample 31 will be formed on the inner part of the cone shaped slide, which is more or less parallel to the axis of rotation 10. As the cone shaped slide is rotated around its own axis 29, liquid sample will wash the whole inner surface, and hereby re-circulation will occur.

As mentioned in the beginning of the application, the invention can be utilised in laboratories for DNA-testing. However, this application does not in any way limit the invention. A slide can contain similar assays for reaction with specific elements or different assays for reaction with a group of elements. The invented process can be utilised for distribution of a sample over any surface of a slide, and the application will then depend on the slide used in the process.

Thus, a portable apparatus for testing purposes on location is a possibility. This could be an apparatus testing for diseases when consulting a doctor, or an apparatus testing for cultures of bacteria in watercourses. Only the slide used in the process defines the application for a given apparatus.

1-4. (canceled)

5. A process for centrifugal distribution of liquid physiological specimens over a surface with an array of assays, said process comprising the steps of: placing said array in a rotating device, distributing said liquid physiological specimens to said surface by the dynamic forces of the rotation, said surface being pointed towards the axis of rotation, forcing the liquid physiological specimens toward said surface under the influence of the dynamic forces of the rotation, said liquid physiological specimens being applied as drops to an area of said surface opposite a drain end, the distribution of said liquid physiological specimens being controlled by adjusting the position of said surface relative to said axis of rotating, in such a way that a thin film of said liquid physiological specimens will be formed over the whole of said surface before drops are forced over the edge in the drain end of the surface.

6. A process for centrifugal distribution of liquid physiological specimens over a surface with an array of assays, said process comprising the steps of: placing said array in a rotating device, distributing said liquid physiological specimens to said surface by the dynamic forces of the rotation, said surface being pointed towards the axis of rotation, the liquid physiological specimens being forced towards said surface under the influence of the dynamic forces of the rotation, wherein the distribution of said liquid physiological specimens is controlled by varying the position or orientation of said surface relative to said axis of rotation, in such a way that a thin film of said liquid physiological specimens is formed on at least varying parts of said surface, and in such a way that said thin film is re-circulated across said array of assays.

7. A process in accordance with claim 6, wherein said control of said surface is caused by a rotating movement around an axis other than that of the rotating device.

8. A process in accordance with claim 6, wherein said surface, to which said liquid physiological specimens are applied, is formed as a hollow in a material part, said hollow forming a closed container when the material part is covered with a lid, and said liquid physiological specimens being applied to said surface before said lid covers said hollow.
